(19) United States
(12) Patent Application Publication
(10) Pub. No.: US 2007/0043427 A1
(43) Pub. Date: Feb. 22, 2007

(54) Lumen-supporting stents

(75) Inventor: Michael J. Lee, Santa Rosa, CA (US)

Correspondence Address: PRESTON GATES & ELLIS LLP;ATTN: C. RACHAL WINGER, 925 FOURTH AVE, SUITE 2900, SEATTLE, WA, 98104-1158, US

(73) Assignee: Medlogics Device Corporation, Santa Rosa, CA (US)

(21) Appl. No.: 11/208,086

(22) Filed: Aug. 19, 2005

(51) Int. Cl. A61F 2/06

(52) U.S. Cl. 623/1.15

(57) ABSTRACT

Disclosed herein are stents manufactured to include increased surface area for bioactive material deposition or enhanced radiopacity at various locations along the length of the stent. The stents of the present invention also can also be manufactured to create a less abrupt transition between stented and unstented portions of a vessel. A less abrupt transition is created by providing increased surface area for bioactive material deposition at the ends of the stent and/or providing end sections (and sections adjacent thereto) of the stents that are more flexible than more centrally located sections of the stent.

### FIELD OF THE INVENTION

This invention relates to an improved implantable stent for the treatment, inhibition and/or prevention of restenosis in coronary or peripheral vessels in humans. More specifically, the invention relates to an improved implantable stent that provides increased surface area for bioactive material deposition and enhanced radiopacity and also can provide for a less abrupt transition between stented and unstented portions of a vessel.

### BACKGROUND OF THE INVENTION

Cardiovascular disease, including atherosclerosis, is the leading cause of death in the United States. The medical community has developed a number of methods and devices for treating coronary heart disease, some of which are specifically designed to treat the complications resulting from atherosclerosis and other forms of coronary artery narrowing.

An important development for treating atherosclerosis and other forms of coronary narrowing is percutaneous translumenal coronary angioplasty, hereinafter referred to as “angioplasty.” The objective of angioplasty is to enlarge the lumen (inner tubular space) of an affected coronary artery by radial hydraulic expansion. The procedure is accomplished by inflating a balloon within the narrowed lumen of the affected artery. Radial expansion of the affected artery occurs in several different dimensions, and is related to the nature of the plaque narrowing the lumen. Soft, fatty plaque deposits are flattened by the balloon, while hardened deposits are cracked and split to enlarge the lumen. The wall of the artery itself is also stretched when the balloon is inflated.

Unfortunately, while the affected artery can be enlarged thus improving blood flow, in some instances the vessel re-occludes chronically (“restenosis”), or closes down acutely (“abrupt reclosure”), negating the positive effect of the angioplasty procedure. Such events of restenosis or abrupt reclosure frequently necessitate repeat angioplasty or open heart surgery. While such restenosis or abrupt reclosure does not occur in the majority of cases, it occurs frequently enough that such complications comprise a significant percentage of the overall failures of the angioplasty procedure, for example, twenty-five to thirty-five percent of such failures.

To lessen the risk of restenosis and abrupt reclosure, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent provides a scaffold which overcomes the natural tendency of the vessel walls of some patients to restenose or undergo reclosure, thus maintaining the openness of the vessel and resulting blood flow.

While stents and stent applications of the type described have been found to work well in a number of patients, there is still room for improvement. For example, some stents release bioactive materials, such as drugs, to reduce the risk of restenosis and abrupt reclosure. These stents have been somewhat successful at aiding in the treatment, inhibition and/or prevention of restenosis or abrupt reclosure. However, the small size and intricate design and configuration of stents has limited the amount of bioactive materials (and therefore the choice of bioactive materials) that can be successfully loaded onto the device. Thus, a need exists for stent designs and configurations that increase a stent's ability to carry and release bioactive materials at a treatment site. Further, as stents are presently used, there can be an abrupt transition between the area of a vessel that is contacted by the stent (and thus receiving the benefits of the stent) and those portions of the vessel that are not. This abrupt transition between stented and unstented portions of a vessel can exacerbate the physiological trauma found at the treatment site. Thus, a need also exists for a stent that provides for a less abrupt transition between stented and unstented portions of a vessel. According to these above-identified problems, the stents of the present invention provide designs and configurations that increase surface area for bioactive material deposition and subsequent release at the treatment site. The stents of the present invention also can provide for less abrupt transitions between stented and unstented portions of a vessel.

### SUMMARY OF THE INVENTION

The present invention provides solutions to problems identified with currently-available stents. First, the stents of the present invention incorporate novel designs and configurations that increase the available surface area of the stent for bioactive material deposition so that increased amounts of bioactive materials are delivered to treatment sites to aid in the treatment, inhibition and/or prevention of restenosis or abrupt reclosure. The stents of the present invention also can be configured to create a less abrupt transition between a stented and unstented portion of a vessel.

As described herein, the abruptness of the transition between stented and unstented portions of a vessel can be caused by both the physical end of the stent itself and the abrupt end of bioactive material release beyond the ends of the stent. Relating to bioactive material release, for example, a portion of a vessel found within the mid-portion of a stent is affected by bioactive material at its immediate location and by release of bioactive materials both slightly before and slightly after its immediate location. A portion of a vessel located at the end of a stent, however, receives less benefit from bioactive material release because, if on the proximal end of the stent (as related to blood flow), it only receives the benefit of bioactive material release at its immediate location and perhaps immediately thereafter, and if on the distal end of the stent, it only receives the benefit from its immediate location and the area directly preceding it.

Thus, the present invention can create a less abrupt transition between stented and unstented portions of vessels by providing (i) increased surface area for bioactive material deposition at the ends of a stent to provide for increased delivery of the bioactive material at these transition sites; and (ii) providing stents with sections at or near the ends of the stent that are more flexible than other portions of the stent by giving these sections less circumferential strength. Stent sections with less circumferential strength can be created by using (i) softer alloys to create these sections of the stent; (ii) thinner (in a radial direction) and/or narrower (in a circumferential direction) structural members (crown and/or struts) at these sections of the stent; (iii) a heat treatment on these stent sections; or (iv) by increasing the length of these stent sections. Alternatively, portions of the stent could be made more flexible by cold working less flexible portions of the stent. Importantly, while increased surface area for bioactive material deposition and sections with less circumferential strength can be provided at the ends of the stent to provide for a less abrupt transition, the present invention is not restricted to this placement. Indeed, increased surface area and areas with less circumferential strength (i.e. more flexibility) can be provided along the entire length of the stent or in discrete areas or points along its length depending on particular treatment objectives.

For example, at a vessel bifurcation, it can be useful to include increased surface area for enhanced radiopacity near the lengthwise midpoint of a stent to position this area at, before, or after the area of the bifurcation. Likewise, at a vessel bifurcation it can be useful to have a stent with enhanced flexibility characteristics near the lengthwise midpoint of the stent so that the stent can better follow the direction of this portion of the vessel. Thus, while the present invention provides for increased surface area for bioactive material deposition and/or increased flexibility at or near the ends of a stent to provide for less abrupt transitions between stented and unstented portions of a vessel, aspects of the present invention can also be adopted at different portions of a stent to serve other important objectives.

In one embodiment of the stents of the present invention, the stent comprises a first end, a second end, and a body wherein the body comprises struts and crowns and wherein each strut is associated with two crowns and wherein the first end comprises crowns and the second end comprises crowns and wherein at least one strut is wider than said strut's corresponding crowns.

In another embodiment of the stents of the present invention, at least one strut associated with a crown of the first end is wider than that strut's corresponding crowns.

In another embodiment of the stents of the present invention, the stent comprises a first end, a second end, and a body wherein the body comprises struts and crowns and wherein each strut is associated with two crowns and wherein the first end comprises crowns and the second end comprises crowns and wherein at least one crown in the first end, the second end or the body further comprises a tab. In another embodiment, the at least one crown that comprises a tab is at the first or the second end of the stent.

In another embodiment of the stents of the present invention, the tabs extend inward towards the lengthwise midpoint of the stent. In another embodiment of the stents of the present invention, the tabs extend outward, away from the lengthwise midpoint of the stent. In another embodiment of the stents of the present invention, every third crown of the first end comprises a tab. In another embodiment of the stents of the present invention, every second crown of the first end comprises a tab. In another embodiment of the stents of the present invention, every crown of the first end comprises a tab. In another embodiment of the stents of the present invention, at least one crown on the first end comprises a tab and every third crown of the second end comprises a tab. In another embodiment of the stents of the present invention, at least one crown on the first end comprises a tab and every second crown of the second end comprises a tab. In another embodiment of the stents of the present invention, at least one crown on the first end comprises a tab and every crown of the second end comprises a tab.

In another embodiment of the stents of the present invention, the stent comprises a first end, a second end, and a body, wherein the body comprises struts and crowns and wherein each strut is associated with two crowns and wherein the first end comprises crowns and the second end comprises crowns and wherein at least one crown and/or its associated strut is more flexible than the majority of crowns and struts in the stent. In another embodiment, the at least one crown and/or its associated strut that is more flexible than the majority of crowns and struts in the stent is found within the first end of the stent. In another embodiment, the at least one crown and/or its associated strut that is more flexible than the majority of crowns and struts in the stent is found within the first end of the stent or the section adjacent thereto. As described supra, more flexible crowns and/or struts of the present invention can be made by using (i) softer alloys to create these portions; (ii) materials that are thinner (in a radial direction) and/or narrower (in a circumferential direction) to construct the portions; (iii) a heat treatment on these portions; or (iv) by increasing the length of the portions of the stent. Alternatively, portions of the stent could be made more flexible by cold working less flexible portions of the stent.

In another embodiment of the stents of the present invention, the more flexible crowns and/or struts are more flexible due to being thinner in a radial direction. In another embodiment of the stents of the present invention, the more flexible crowns and/or struts are more flexible due to being narrower in a circumferential direction. In another embodiment of the stents of the present invention, the more flexible crowns and/or struts are more flexible due to being thinner in a radial direction and narrower in a circumferential direction. In these embodiments of the present invention, crowns alone can be more flexible, struts alone can be more flexible, or associated crowns and struts can be more flexible.

In another embodiment of the stents of the present invention, the stent comprises a first end, a second end, and a body, wherein the body comprises struts and crowns and wherein each strut is associated with two crowns and wherein the first end comprises crowns and the second end comprises crowns and wherein at least one crown and/or its associated strut is more flexible than the majority of crowns and struts in the stent and wherein at least one other strut in the body of the stent is wider than that strut's corresponding crowns.

In another embodiment of the stents of the present invention, at least one crown at the first end and/or its associated strut is more flexible than the majority of crowns and struts in the stent and at least one crown at the second end and/or its associated strut is more flexible than the majority of crowns and struts in the stent.

In another embodiment of the stents of the present invention, the stent comprises a first end, a second end, and a body, wherein the body comprises struts and crowns and wherein each strut is associated with two crowns and wherein the first end comprises crowns and the second end comprises crowns and wherein at least one crown at the first end and/or its associated strut is more flexible than the majority of crowns and struts in the stent and wherein at least one crown further comprises a tab.

In one embodiment of the stents of the present invention, the stent comprises a first end, a second end, and a body wherein the body comprises struts and crowns and wherein each strut is associated with two crowns and wherein the first end comprises crowns and the second end comprises crowns and wherein at least one strut is wider than said strut's corresponding crowns and wherein at least one crown further comprises a tab.

Another embodiment of the present invention includes methods of making a stent wherein the method comprises providing a stent comprising a first end, a second end, and a body wherein the body comprises struts and crowns and wherein each strut is associated with two crowns and wherein the first end comprises crowns and the second end comprises crowns and wherein at least one strut is wider than the strut's corresponding crowns and further providing at least one crown that comprises a tab.

### BRIEF DESCRIPTION OF THE DRAWINGS

FIG. 1A shows a “wide strut” stent configuration that provides increased surface area for bioactive material deposition along the entire length of the stent. FIG. 1B shows a different wide strut stent configuration in its compressed form for delivery.

FIGS. 2A and 2B depict a wide strut configuration of the present invention including one embodiment of the tabs of the present invention. This embodiment is depicted after circumferential expansion.

FIGS. 3A and 3B depict a wide strut embodiment of the present invention with an alternative embodiment of the tabs of the present invention. This embodiment is depicted after circumferential expansion.

FIGS. 4A and 4B depict a wide strut embodiment of the present invention with an alternative embodiment of the tabs of the present invention. This embodiment is depicted after circumferential expansion.

FIGS. 5A and 5B show one embodiment of the present invention using both tabs to increase surface area for bioactive material deposition and thinner and narrower end crowns to provide a less abrupt transition from a stented to unstented portion of a vessel. This embodiment is depicted after circumferential expansion and does not include a wide strut configuration.

FIG. 6 shows an embodiment of the present invention including the use of two different tab embodiments after circumferential expansion.

FIG. 7 shows an alternative wide strut tabbed embodiment of the present invention after circumferential expansion.

### DETAILED DESCRIPTION OF THE INVENTION

U.S. Pat. Nos. 5,292,331 and 5,135,536 to Boneau and Hilstead respectively, and the references cited therein, make it clear that stents can be configured and constructed in many different ways. The present invention is applicable to all known stent configurations, and it will be readily apparent from the following discussion of several exemplary configurations how the invention can be applied to any other type of stent construction.

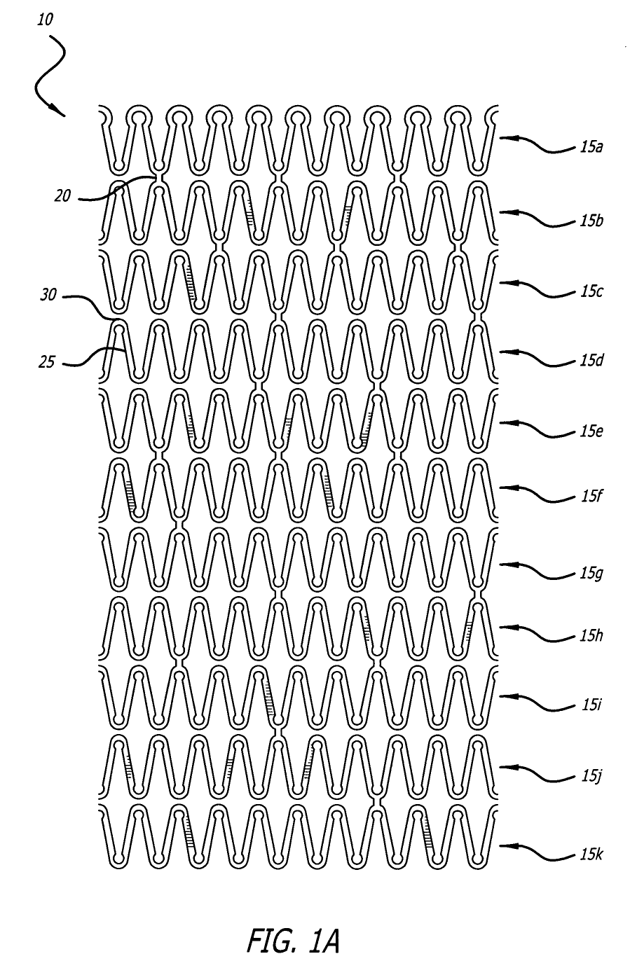

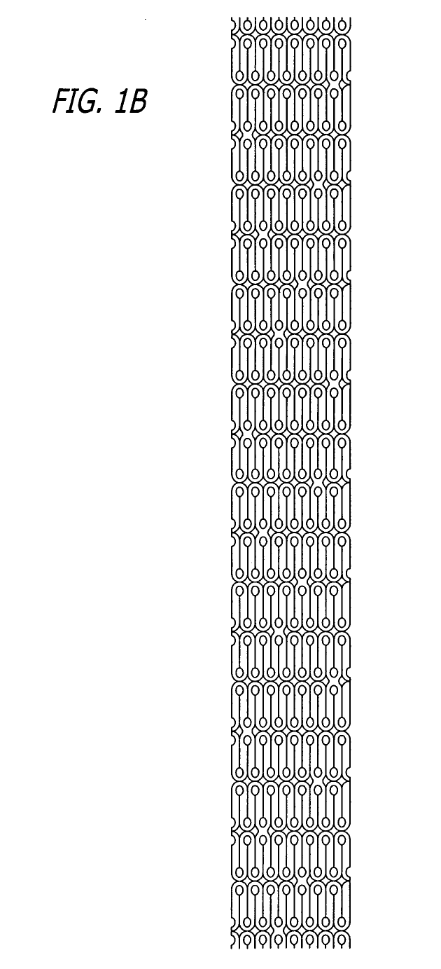

An illustrative stent 10 is shown in FIG. 1A. In FIG. 1A, stent 10 includes 11 “sections,” 15a-k. While these “sections” of the stent are referred to as such for ease of description, one should note that the sections 15 are not formed separately, but instead are continuous and joined by “crossovers” 20. The sections 15 may have more or less undulations than are shown in FIG. 1A (and generally similar subsequent FIGS.), but the simplified depictions shown herein will be sufficient to illustrate the present invention. Further, while this FIG. 1A depicts 11 repeating sections, it is to be understood that the stents of the present invention can include more or less than 11 sections.

As stated earlier, it is often beneficial for an implanted stent to release a bioactive material to reduce the physiological trauma associated with the stent's implantation and to aid in the treatment, inhibition and/or prevention of restenosis or abrupt reclosure. The stents of the present invention are designed to allow for varying amounts of bioactive material release along the stent due to increased surface area of various portions of the stent. As stated earlier, in one embodiment the increased surface area is provided at the ends of the stent to provide more bioactive material release at the ends of a stent and, as a result, a less abrupt transition between stented and unstented portions of a vessel. Increased surface area may also, however, be provided along the entire length of the stent or in discrete regions or points along the stent. One way that the present invention achieves increased surface area for increased bioactive material deposition is by widening the struts 25 of the stent 10 relative to the particular strut's 25 crowns 30, as shown in FIG. 1A. In this FIG. 1A, the struts 25 are widened relative to their associated crowns 30 along the entire length of the stent 10.

FIG. 1B shows a “wide strut” embodiment of a stent of the present invention in a compressed form for delivery. As can be seen in FIG. 1B, the wide strut configuration of the present invention optimizes the use of available space on a stent mounted on a balloon for bioactive material deposition. This optimization increases available surface area for bioactive material deposition without affecting the crossing profile of the stent. Wide strut embodiments of the present invention can adopt many different configurations and dimensions as long as the widened struts are wider than their respective crowns.

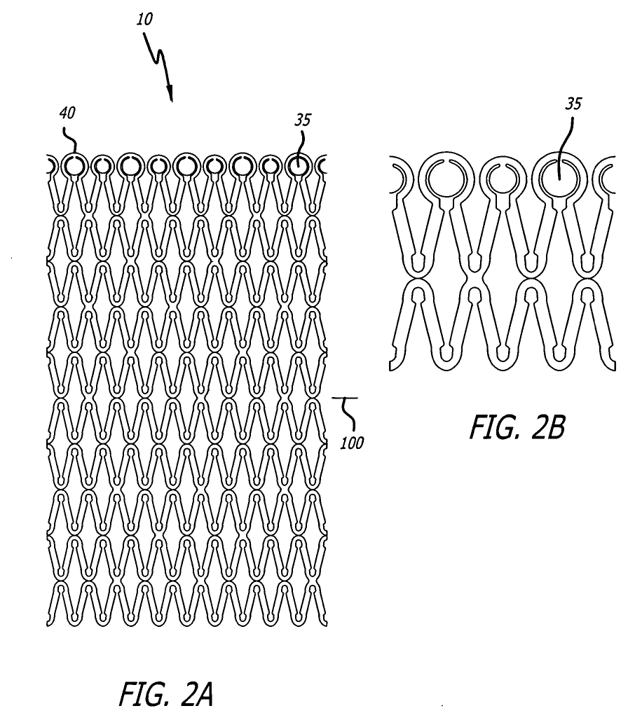

In addition to providing at least one wide strut (relative to crowns) to increase surface area for bioactive material deposition, the present invention also utilizes “tabs” to increase the surface area of a stent. FIGS. 2A and 2B (FIG. 2B is a close-up view of the end of FIG. 2A) depict one embodiment of the tabs 35 of the present invention. As can be seen, in this embodiment, one end of the stent 10, is comprised of enlarged or “bulbed” end crowns 40 with internal tabs 35. The internal tabs 35 are found within the bulbed end crowns 40 and extend inward back towards the lengthwise midpoint 100 of the stent 10. By “extend inwards back towards the lengthwise midpoint” it is not meant that these tabs bend out of the plane of the stent. Instead “extend inwards” means that the tabs are oriented so that, if found on an end crown, they would not increase the length of the stent. Because the increased surface area is located at the ends of the stent where bioactive material release generally abruptly ends, stents incorporating these tabs can provide for a less abrupt transition between stented and unstented portions of a vessel due to the increased release of bioactive materials.

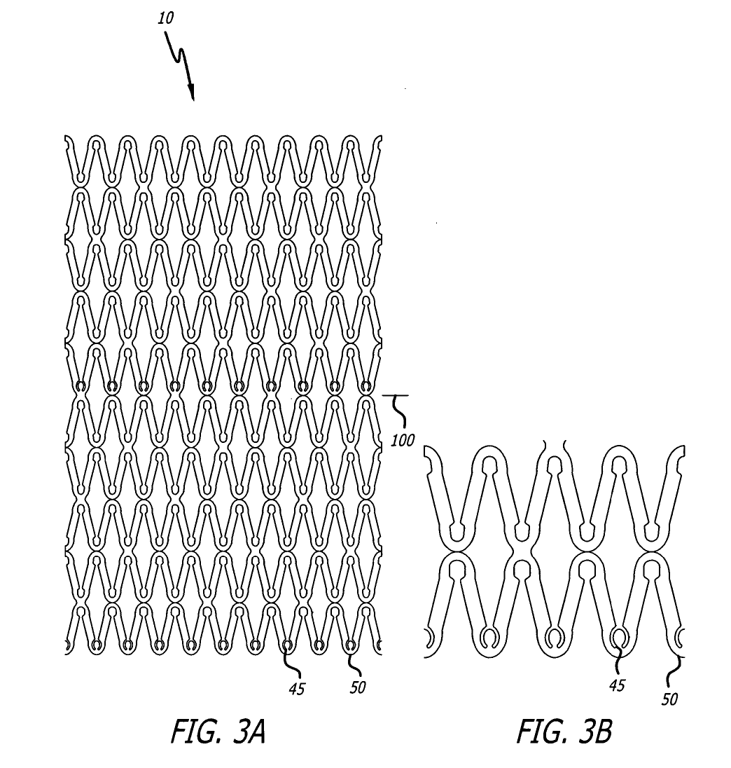

FIGS. 3A and 3B (FIG. 3B is a close-up view of the end of FIG. 3A) also depict tabs 45 that extend inwards from the terminal end crowns 50 back towards the lengthwise midpoint 100 of the stent 10. In this embodiment, however, the crowns 50 are not enlarged or bulbed as they are in the embodiment depicted in FIGS. 2A and 2B. Instead, the end crowns 50 maintain the same dimensions as those found within the body of the stent 10. Again, the tabs depicted within this embodiment of the present invention are found within the end crowns and do not affect the overall length or crossing profile of the stent. FIG. 3A also demonstrates that the tabs of the present invention can be included anywhere along the length of the stent. In this embodiment, tabs are included at the approximate lengthwise midpoint 100 of the stent 10. Again, in this embodiment, tabs are depicted in every crown in the rows of crowns in which they are found. Such inclusion in every crown of a row is not required, however. In some embodiments of the present invention, a row of crowns can have less tabs than crowns including only one tab-containing crown.

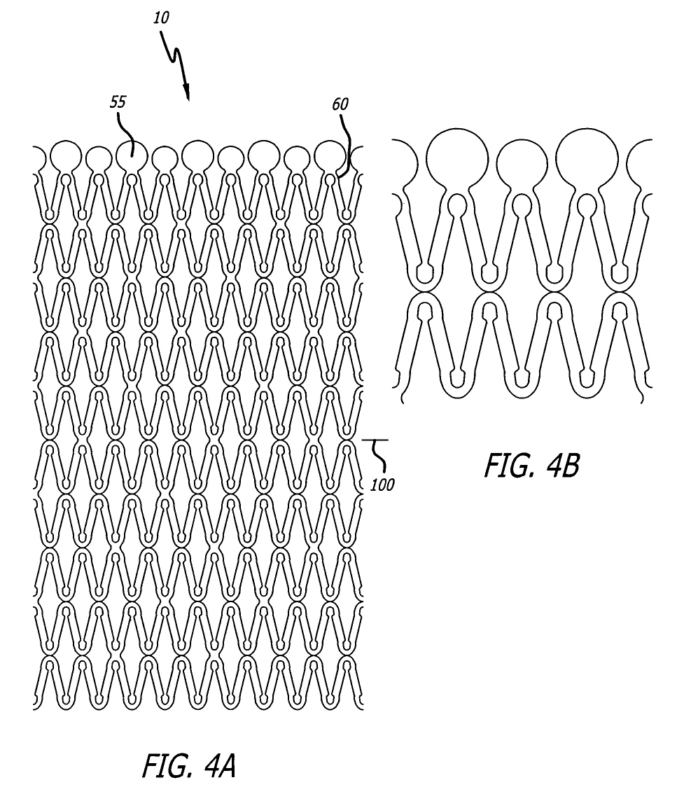

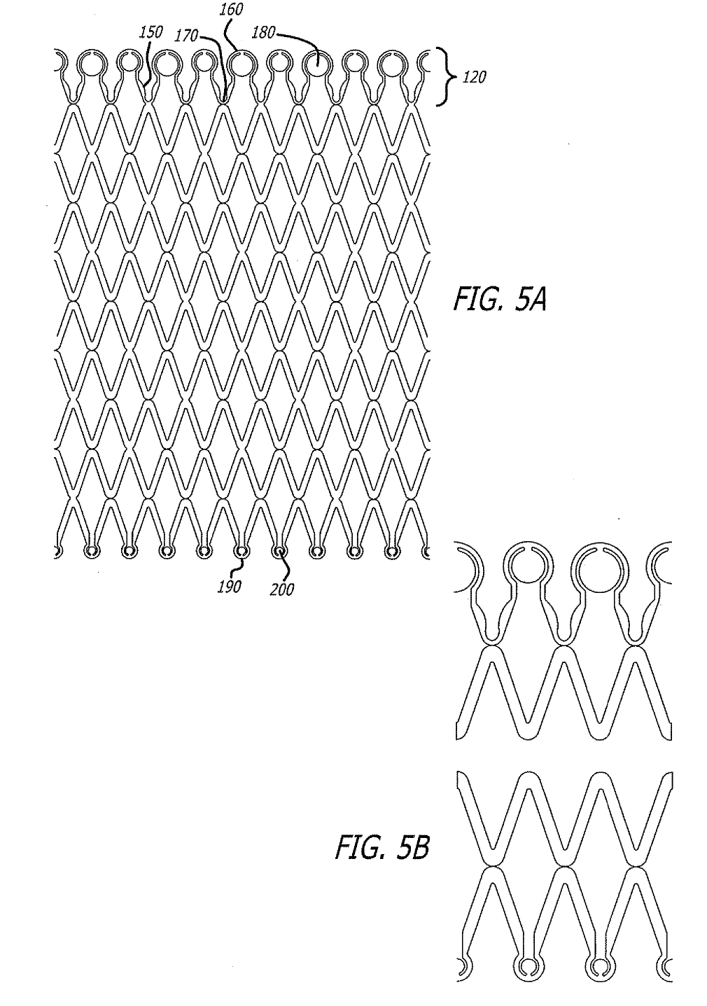

FIGS. 4A and 4B (FIG. 4B is a close-up view of the end of FIG. 4A) depict another wide-strut tabbed embodiment of the present invention. In this embodiment, the tabs 55 extend away from the lengthwise midpoint 100 of the stent 10. By “extend away from the lengthwise midpoint,” it is not meant that these tabs bend out of the plane of the stent. Instead “extend away” means that the tabs are oriented so that, if found on an end crown, they do increase the length of the stent. Specifically, the tabs 55 are continuous with and extend from the ends of the terminal end crowns 60. Thus, these tabs 55 do extend the length of the stent 10. Depending on whether these tabs 55 adopt the same width as the crowns of a particular embodiment, these tabs may or may not affect the crossing profile of the stent 10. If the tabs maintain the same width or have a smaller width than the crowns, the crossing profile will not be affected even if every end crown comprises such a tab. The tabs of the present invention can have widths larger than the crowns of a particular embodiment and, if included on every crown, would affect the crossing profile. Including tabs with widths larger than the width of crowns of a particular embodiment may not affect the crossing profile, however, if such tabs are included on less than every end crown or are configured to overlap with one another when the stent is mounted and crimped on the balloon catheter.

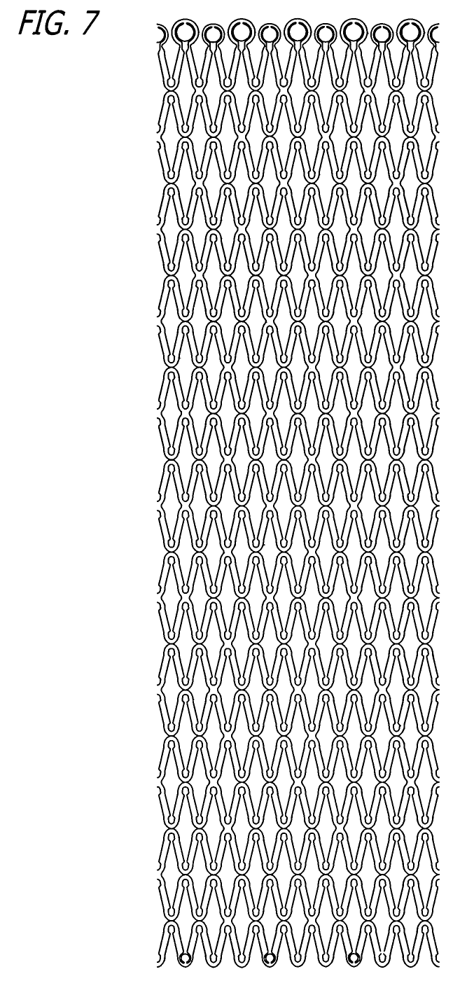

The tabs of the present invention, whether found within or extending from and beyond the end crowns can take on a variety of shapes and sizes. Further, these tabs can be placed at every end crown (see FIG. 6), every other end crown, every third end crown (see FIG. 7), etc. Further, the tabs of the present invention are not restricted to placement within end crowns, but can also be placed within any crown of a stent of the present invention. In addition to providing increased surface area for bioactive material deposition, the tabs and widened struts of the present invention also provide for increased mass of the stent. This increased mass (which is not structurally required) is beneficial for enhanced radiopacity in stent positioning. For example, tabs placed at different portions along the length of a stent can be used to position the stent appropriately in an area of a vessel bifurcation or other specific treatment site.

As stated earlier, embodiments of the present invention can increase the surface area of a stent at any point along the body or end crowns of the stent. Increasing the surface area of a stent for additional bioactive material deposition and subsequent release at one or more ends of a stent may ease the transition between stented and unstented portions of a vessel by providing for additional bioactive materials that combat restenosis at the transition site. In addition to providing for increased bioactive material release at the transition site from stented to unstented portions of a vessel, embodiments of the present invention can include additional features to ease the transition even further. For example, stents of the present invention may include one or more end sections and sections adjacent to end sections that hold open or stent the treated vessel less strongly (i.e. are more flexible) than more centrally located sections of the stent. Flexibility of these end sections and sections adjacent to end sections can be increased by constructing these sections of the stent with softer alloys, by using thinner (in a radial direction) or narrower (in a circumferential direction) material to create these sections or by increasing the length of these sections. Although these sections of the stent are designed to be less strong than other more centrally located sections of the stent, they nevertheless have sufficient radial strength to hold open the vessel, albeit to a lesser degree than the more centrally-located stent sections.

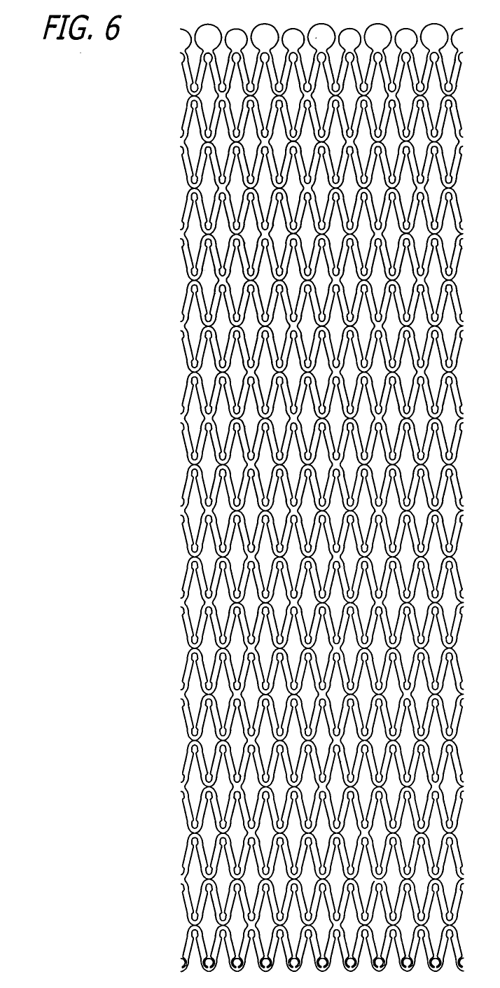

FIGS. 5A and 5B (FIG. 5B is a close-up view of the end of FIG. 5A) provide one embodiment of the present invention that creates a more flexible end section (as compared to more centrally-located sections) to ease the transition from a stented to an unstented portion of a vessel. In this embodiment, the metal (or any material from which the stent is made) of the more flexible end 120 is made thinner (in a radial direction) and narrower (in a circumferential direction) than other areas of the stent, and is therefore more flexible, than the remaining portions of the stent. These characteristics are depicted in FIGS. 5A and 5B as thinner and narrower struts 150 and crowns 160, 170, 190 near the ends of the stent. Optional tabs 180, 200 may also be thinned in a manner similar to their respective crowns.

By providing at least one end section of a stent of the present invention that is more flexible than more centrally-located sections, the transition or transitions between the stented and unstented portions of the vessel are made less abrupt. This feature can reduce the physiological trauma found at treatment sites and thus can reduce the likelihood of adverse reactions at this site. Further, the embodiment depicted in FIG. 5 is not a wide strut embodiment.

Note that in FIG. 5, the end crowns, in addition to the next adjacent crowns, are thinner and narrower in this depicted embodiment. In other embodiments, additional or different crowns and/or struts can be narrowed as needed to create desired flexibility characteristics. Indeed, it should be understood, that any number of crowns and/or struts in any position along the length of the stent can be made more flexible in keeping with the present invention as long as the more flexible crowns and/or struts are kept in a numerical minority as compared to the remaining crowns and/or struts. Stated another way, the majority of crowns and/or struts within a particular stent must not adopt enhanced flexibility characteristics. Majority as used herein is in keeping with the standard dictionary definition of the term meaning, “a number greater than half.”

FIG. 6 and FIG. 7 show that different aspects of the present invention can be put together in various combinations. For example, FIG. 6 depicts a wide strut stent embodiment with one end including outwardly-extending enlarged tabs and the other end including inwardly-extending normal crown sized tabs. FIG. 7 depicts a wide strut stent embodiment with one end including inwardly-extending enlarged tabs and the other end including inwardly-extending normal crown sized tabs. Note that on one end of the stent depicted in FIG. 7, tabs are included within every third crown. Again, these figures are not meant to be limiting but instead are presented to show how various configurations of the stents of the present invention may be combined to achieve particular treatment objectives.

One non-limiting purpose of the stents of the present invention is to maximize stent artery coverage without compromising the crossing profile of the stent. Another non-limiting purpose of the stents of the present invention is to maximize drug elution without compromising the crossing profile of the stent. Another non-limiting purpose of the stents of the present invention is to add surface area to the stent without causing a significant change in the mechanical properties of the stent.

The stents of the present invention can be used in any blood vessel, including, for example and without limitation, the coronary vasculature (which includes without limitation the right, left common, left anterior descending and circumflex arteries and their branches) and the peripheral vasculature (including without limitation branches of the carotid, aorta, femoral, renal, popliteal, and related arteries). While the stents of the present invention mainly have been described in terms of their use in a blood vessels, they can also be used in other lumens of the body, for example and without limitation, respiratory ducts, gastrointestinal ducts, bile ducts, the urinary system, the digestive tube, and the tubes of the reproductive system in both men and women.

It is to be understood that the present invention is not limited to the particular embodiments, materials, and examples described herein, as these can vary. Further, the tabs of the present invention can be made of material that is dissimilar to the material or materials that make up the other portions of the stent. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a stent” or “a tab” is a reference to one or more stents or tabs and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising a first end, a second end, and a body wherein said body comprises struts and crowns and wherein each said strut is associated with two said crowns and wherein said first end comprises crowns and said second end comprises crowns and wherein at least one strut is wider than said strut's corresponding crowns.

2. The stent according to claim 1, wherein said at least one strut has an associated crown that is part of said first end.

3. The stent according to claim 2, further comprising at least one additional strut that is wider than said strut's corresponding crowns and wherein said at least one additional strut has an associated crown that is part of said second end.

4. The stent according to claim 1, further comprising at least one tab in said crown of said first end, said second end or said body.

5. A stent comprising a first end, a second end, and a body wherein said body comprises struts and crowns and wherein each strut is associated with two crowns and wherein said first end comprises crowns and said second end comprises crowns and wherein at least one of said crowns in said body, said first end or said second end comprises a tab.

6. The stent according to claim 5, wherein said tab extends inwards towards said lengthwise midpoint of said stent.

7. The stent according to claim 5, wherein said tab extends outwards, away from said lengthwise midpoint of said stent.

8. The stent according to claim 5, wherein at least one crown of said first end comprises a tab.

9. The stent according to claim 5, wherein at least every third crown of said first end comprises a tab.

10. The stent according to claim 9, wherein at least every second crown of said first end comprises a tab.

11. The stent according to claim 10, wherein every crown of said first end comprises a tab.

12. The stent according to claim 8, wherein at least every third crown of said second end comprises a tab.

13. The stent according to claim 12, wherein at least every second crown of said second end comprises a tab.

14. The stent according to claim 13, wherein every crown of said second end comprises a tab.

15. A stent comprising a first end, a second end, and a body, wherein said body comprises struts and crowns and wherein each strut is associated with two crowns and wherein said first end comprises crowns and said second end comprises crowns and wherein at least one crown and/or its associated strut(s) are more flexible than the majority of said crowns and said struts within said stent.

16. The stent according to claim 14, wherein said at least one more flexible crown and/or strut(s) is more flexible than said majority of said crowns and said struts within said stent due to being thinner in a radial direction.

17. The stent according to claim 14, wherein said at least one more flexible crown and/or strut(s) is more flexible than said majority of said crowns and said struts within said stent due to being narrower in a circumferential direction.

18. The stent according to claim 14, wherein said at least one more flexible crown and/or strut(s) is more flexible than said majority of said crowns and said struts within said stent due to being thinner in a radial direction and narrower in a circumferential direction.

19. The stent according to claim 14, wherein said at least one more flexible crown and/or strut(s) is more flexible than said majority of said crowns and said struts within said stent due to being constructed of a softer alloy.

20. The stent according to claim 14, wherein said at least one more flexible crown and/or strut(s) is more flexible than said majority of said crowns and said struts within said stent because said more flexible crown and/or strut(s) have a greater length than said majority of said crowns and said struts.

21. The stent according to claim 14, further comprising at least one strut in said body that is wider than said strut's corresponding crowns.

22. The stent according to claim 14, wherein said at least one crown and/or its associated strut(s) is a crown within said first end of said stent.

23. The stent according to claim 14, further comprising at least one tab in a crown of said first end, said second end or said body.
